(19) United States
(12) Patent Application Publication
(10) Pub. No.: US 2009/0286720 A1
(43) Pub. Date: Nov. 19, 2009

(54) PHARMACEUTICAL COMPOSITION FOR THE DIAGNOSIS, PREVENTION OR TREATMENT OF A TUMORAL PATHOLOGY COMPRISING AN AGENT MODULATING THE POLYMERIZATION STATE OF ACTIN

(75) Inventors: Christian Auclair, Paris (FR); Valerie Amsellem, Paris (FR); Martial Hervy, Paris (FR); Frederic Subra, Paris (FR)

Correspondence Address: HARNESS, DICKEY & PIERCE, P.L.C., P.O. BOX 828, BLOOMFIELD HILLS, MI, 48303, US

(73) Assignees: Bioalliance Pharma, Paris (FR); Ecole Normale Superieure de Cachan, Cachan (FR); Institut Gustave Roussy - IGR, Villejuif Cedex (FR); Centre National de la Recherche Scientifique - CNRS, Paris Cedex 16 (FR)

(21) Appl. No.: 12/426,833

(22) Filed: Apr. 20, 2009

Related U.S. Application Data
(62) Division of application No. 10/740,266, filed on Dec. 18, 2003.
Application No. 10/740,266 is a continuation of application No. PCT/FR02/02106, filed on Jun. 18, 2002.

(30) Foreign Application Priority Data
Jun. 18, 2001 (FR) 01/07976

(51) Int. Cl. A61K 38/15; A61P 35/00

(52) U.S. Cl. 514/11

(57) ABSTRACT

A pharmaceutical composition for the treatment, prevention or diagnosis of a tumoral pathology comprising an active agent which stabilizes an actin network of a cellular cytoskeleton.

### CROSS-REFERENCE TO RELATED APPLICATIONS

This application is a divisional of U.S. patent application Ser. No. 10/740,266, filed Dec. 18, 2003, which is a continuation of International Application No. PCT/FR02/02106, with an international filing date of Jun. 18, 2002 (now WO 02/102846, published Dec. 27, 2002), which is based on French Patent Application No. 01/07976, filed Jun. 18, 2001.

### INTRODUCTION

This invention relates to the field of cancer treatment and prevention. It is based on the modification of certain cell phenotype characteristics linked to the structure of the cytoskeleton such as adherence and motility when cells evolve into a tumor phenotype.

### SUMMARY

This invention relates to a pharmaceutical composition for the treatment, prevention or diagnosis of a tumoral pathology including an active agent which stabilizes an actin network of a cellular cytoskeleton.

This invention also relates to a vector of nonviral intracellular transport associated with the active agent associated with a vector of intracellular transport.

This invention further relates to a vector of intracellular transport including a vector of viral recombinant expression including a cDNA coding for a zyxin gene or a functional fragment thereof.

This invention still further relates to a nonhuman transgenic mammal including at least one genetically modified cell underexpressing a zyxin gene or a functional fragment thereof.

This invention yet further relates to a method of identifying compounds that stabilize an actin network of a cytoskeleton of a cell including detecting a phenotypic reversion of expression of zyxin induced by the compounds including contacting a compound to be tested with the cell, and quantifying expression of zyxin in the cell.

This invention also further relates to a method for diagnosing a tumoral pathology including obtaining cells from a patient, and quantifying expression of zyxin in the cells.

This invention still yet further relates to a method of analyzing a tumor phenotype of a patient including collecting cells from a patient at different intervals, quantifying expression of zyxin in the cells collected at different intervals, comparing levels of expression and constructing a phenotypic differential profile of the patient.

This invention yet again relates to the method of analyzing a tumor phenotype of a patient, wherein the intervals correspond to different periods during antitumor treatment of a patient and wherein quantifying the expression of zyxin is performed by comparing expression of messenger RNA in the cells collected at intervals.

This invention finally also relates to a method of treating or preventing hepatocarcinomas, a method of treating or preventing mesenchymal tumors, a method of treating or preventing neuroectodermal cancer and a method of treating or preventing Ewing's sarcoma, each method including administering a therapeutically effective amount of the composition for the treatment, prevention or diagnosis of a tumoral pathology including an active agent which stabilizes an actin network of a cellular cytoskeleton to a patient in need thereof.

### DRAWINGS

Other advantages of the invention will become apparent from the experimental studies carried out by the applicant and described in the “Material and Methods” section below with reference to the attached figures in which:

FIG. 1 is a diagram of the interactions between zyxin and its cellular partners at the level of the plasma membrane.

FIG. 2 is a diagram of the construction of the viral transport vector associated with zyxin.

FIG. 3 shows the images of the cellular structures revealed specifically. FIG. 3A shows the actin filaments revealed by marking the cells with a phalloidin probe coupled with FITC. FIG. 3B shows the localization of zyxin revealed by marking of cells with an anti-zyxin antibody revealed with a murine anti-IgG antibody coupled with TRITC.

FIG. 4A illustrates Northern blot results with the detection of zyxin mRNA from 10 μg of total RNA using the 250-pb human zyxin probe.

FIG. 4B is a schematic representation of the retroviral shuttle containing the zyxin reading frame of the mRNA containing the open reading frame of human zyxin and neo/CMV probes and zyxin used for the development of the Northern blots.

FIG. 5 illustrates Northern blot results with the detection of mRNA stemming from LTR 5′, from 10 μg of total RNA deposited on denaturing agarose gel by the neo/CMV 1081-pb probe.

FIG. 6 shows Western blot and immunodetection images of the zyxin protein in the different cell lines from 60-μg protein extracts stemming from different cell lines.

FIG. 7 illustrates Western blot images obtained after immunoprecipitation of the protein extracts of the protein EWS-FLI-1 extracted from zyxin E-F clones.

FIG. 8A represents an antisense sequence (SEQ ID NO: 4).

FIG. 8B is a diagram of the construction of the retroviral shuttle expressing the antisense against zyxin AUG.

FIG. 8C shows the detection by RT PCR of the antisense RNA directed against zyxin AUG.

FIG. 9 shows Western blot and immunodetection images of zyxin protein from 40-μg of protein extract stemming from different cell lines.

FIG. 10 is a graphic representation of the comparative study of the variations of the expression levels of the genes performed by macroarrays between the line NIH3T3 and the lines EWS-FLI, as zyxin 1 and as zyxin 2.

FIG. 11 illustrates the in vitro measurement of actin polymerization by fluorescence anisotropy.

FIGS. 12A to 12G show the study of the results of the study of the morphological modification of the cells NIH3T3 and EWS-Fli in the presence of dolastatin (D11): untreated NIH3T3 cells (FIG. 12A); NIH3T3 cells transfected by EWS-Fli (FIGS. 12B, 12C); NIH3T3 cells transfected by EWS-Fli incubated in the presence of 10 μM (FIG. 12D) or 100 μM (FIG. 12E) of jasplakinolide or incubated with 10 μM (FIG. 12F) or 20 μM (FIG. 12G) of D11.

FIG. 13 shows the results of the study of the toxicity of dolastatin 11 (D11) on the NIH3T3 and EWS/Fli cells.

FIG. 14 shows the results of the study of the development of tumors in a nude mouse in the presence of dolastatin 11.

FIG. 15 shows the results of the study of the displacement rate of the cells by phase-contrast microscopy.

FIG. 16A shows the chemical structure of dolastatin 11.

FIG. 16B shows the chemical structure of jasplakinolide.

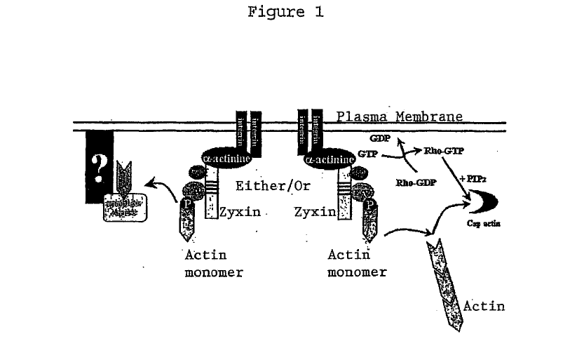

### DETAILED DESCRIPTION

Tumor phenotype characteristics are linked to the structure of the cytoskeleton of cells, the stability of which is ensured by polymerization of the actin networks that constitute it. This invention is based on the correlation between the underexpression of genes intervening in the stabilization of the actin network of the cytoskeleton of cells, such as, for example, the underexpression of the zyxin gene and the phenotypic transformation of a normal phenotype versus a tumor phenotype of said cells.

The invention includes administration of a pharmaceutical composition which stabilizes the actin network of the cytoskeleton such as, for example, compounds inducing the overexpression of the zyxin gene in a cell comprising a tumor phenotype and induces the phenotypic reversion of the cell into a normal phenotype.

Formation of the structure of the cytoskeleton via the dynamic of the polymerization of actin plays an essential role in the maintenance of the invasive tumor phenotype and, consequently, a pharmacologic action having as a consequence an augmentation of the amount of actin F in the stationary state in a tumor cell constitutes a means for diminishing the invasive character of the malignant cell, or even reestablishing a normal phenotype.

Thus, this invention identifies compounds that modulate the polymerization state of actin and uses the compounds for drugs useful for the diagnosis, prevention and/or treatment of tumor pathologies.

Such compounds that stabilize the actin network can be, for example, inhibitors of cofilin which is an enzyme known for its action on the depolymerization mechanism of actin F. In its active form (dephosphorylated cofilin), it induces the rupture of helices and promotes depolymerization of actin F.

Identification of genes involved in tumoral transformation and maintenance of the malignant phenotype is one of the elements required for the development of new therapeutic approaches to tumor pathologies. The pertinent experimental approaches require the availability of biological materials constituted notably by couples of the type “normal tissue/tumoral tissue”. These couples enable the implementation of differential expression measures.

However, these approaches have drawbacks because the relevance of the results depends on the significant character of the couples employed. The lines should have a clearly characterized phenotype. They should have a nontumoral immortal phenotype and the malignant transformation of the immortal lines must be induced by a unique genetic event. Finally, it should be possible to easily obtain phenotypic revertants.

The most pertinent models are provided by the tumor phenotypes induced by oncogenous fusion proteins such as BCR-Ab1, PML-RAR which lead to leukemia phenotypes as well as EWS-Fli-1, responsible for Ewing's sarcoma.

Ewing's sarcoma is a tumor of neuroectodermal origin which is characterized by a chromosomal translocation involving the band q12 of the reworked chromosome 22 with band q24 of chromosome 11: t(11; 22) (q24; q12) (Turc-Carel et al., 1984) leading to the formation of a chimera gene associating the proto-oncogene EWS with a member of the family of ETS genes. The breaking points associated with the majority translocation t(11; 22) are located in a 7-kb region belonging to the EWS gene for chromosome 22 and a 50-kb region belonging to FLI-1 for chromosome 11 (Zucman et al., 1993). The result of this chromosomal translocation generates a derivative of chromosome 22 in which part 5′ of the gene EWS is associated with part 3′ of the gene FLI-1 (Delattre et al., 1992).

The fusion gene expresses the chimera protein EWS-FLI-1 possessing oncogenous properties. Thus, the chimera protein EWS-Fli-1 is capable of transforming murine fibroblasts of type NIH3T3 in culture (Ohno et al., 1993) and inducing tumors in nude mice. The association of the N-terminal domain of EWS and the C-terminal domain of FLI-1 is necessary for its transformant power (May et al., 1993).

We developed a model constituted, on the one hand, by nontumoral, immortal, normal murine NIH3T3 fibroblasts and, on the other hand, by fibroblasts expressing the fusion protein EWS-Fli-1 in a constitutive manner and having a tumor phenotype in nude mice. This cell couple makes possible an evaluation of the differential expression characterizing the acquisition of the tumor phenotype.

This evaluation was performed using the cDNA micro-array from Clontech which can evaluate simultaneously the expression of 588 genes.

In a second step, stable phenotypic revertants were obtained by infecting the cells transformed by retroviral vectors coding for antisense RNA directed against the oncogenous fusion gene. A second cell couple (tumor cells/nontumoral revertant cells) was thereby obtained and was the object of differential expression.

It was possible to identify the genes, the variation of expression of which is linked to the expression of the oncogenous protein and to the nature of the phenotype, to provide new means for the treatment, prevention or diagnosis of cancers.

Immunostaining of the actin filaments on fibroblasts showed that the malignant transformation of the fibroblasts mediated by a chimera protein EWS-Fli-i is translated by a profound modification of the morphology of the fibroblasts with, notably, a decrease in the focal points. This is accompanied by a remodeling of the cytoskeleton and notably of the polymerized actin networks.

In particular, the tumor phenotype is influenced by the level of expression of zyxin and underexpression of the zyxin gene is a sufficient condition for transforming a normal fibroblast into a tumorigenic fibroblast.

It is known that zyxin is a protein comprising LIM domains present in the focal adherence plaques of the fibroblasts and lamellipodia of the superior eukaryote cells. These LIM motifs, in the form of zinc fingers, are implicated in the interactions of the protein-protein type (Scheimechel et al. 1994. “The LIM domain, a new structural motif found in zinc-finger-like proteins”. Trends Genet. 10: 315-320.

Zyxin is implicated in the regulation of the polymerization of actin filaments and has structural and functional properties in common with ActA of Listeria (Golsteyn et al., 1997). Zyxin is believed to act as an anchoring intermediary between the plasma membrane via .alpha.-actinin and the integrins and actin filaments. It is clearly implicated in the architecture of the cell cytoskeleton, adherence and motility (Crawford and Beckerle, 1991). Structurally, zyxin comprises a proline-rich N-terminal region, a nuclear exportation signal (NES) peptide as well as regions rich in amino acids histidine and cysteine forming the LIM motifs in the C-terminal part (Sadler et al., 1992. “Zyxin and cCRP: Two interactive LIM domain proteins associated with the cytoskeleton”. J. Cell. Biol. 119: 1573-1587).

The mechanism of zyxin-dependent tumorigenesis implicates modifications of motility, adherence and signalization linked to cell-cell and extracellular cell-matrix interactions.

Analysis of differential expression profiles verified that the dependent morphological modifications of the expression of an EWS-Fli-1 chimera protein are correlated with the variations of expression of the zyxin gene.

We thereby confirmed that the diminution of expression of the zyxin gene, implicated in the stabilization of the actin network intervening in the organization of the cell cytoskeleton, is directly linked to the acquisition and maintenance of the tumor phenotype and that the induction of the overexpression of the genes leads to the reversion of the tumor phenotype.

Thus, this invention provides novel pharmaceutical compositions for the treatment and prevention of cancer comprising compounds that stabilize the actin network of the cell cytoskeleton. Compositions according to the invention are capable of restoring a nontumoral phenotype unlike the prior art which destroy the cells and are thus likely to cause side effects.

We developed a method for the identification of antitumor compounds that stabilize the actin network of the cell cytoskeleton based on the detection of the reversion of the tumor phenotype linked to the expression of zyxin.

This invention, thus, includes a pharmaceutical composition for the treatment, prevention or diagnosis of a tumoral pathology comprising an active agent stabilizes the actin network of the cytoskeleton of a cell, selected from the group comprising: the zyxin protein, a nucleic acid molecule comprising or constituted of the zyxin gene, a fragment thereof or their complementary sequence, or an antisense nucleic acid thereof, a cell or a set of cells overexpressing the zyxin gene or a protein coded for a fragment thereof, and an inhibitor of cofilin.

### DEFINITIONS

As sometimes used herein, the term “zyxin fragment” means any polypeptide fragment of zyxin capable of conserving the biological function of zyxin and particularly its function of stabilizing the actin network of the cytoskeleton.

The term “fragment of the zyxin gene” means any nucleic acid fragment of zyxin and, in particular, the cDNA of the gene coding for zyxin and/or the different functional domains thereof, such as those coding for: the N-terminal region of the protein rich in proline, the nuclear exportation signal (NES), and the regions forming the LIM motifs in the C-terminal part.

The term “derivative of the zyxin gene” means any nucleic acid modified chemically or by genetic recombination, but conserving the function of the gene, notably its capacity to code for a polypeptide when it is expressed in a suitable host, which conserves the biological functions of the zyxin protein and in particular its function of stabilizing the actin network of the cell cytoskeleton. Such modifications comprise, for example, the modification, addition or suppression of bases by fusion with other nucleic acids such as, for example, regulator elements or chimera molecules comprising heterologous cDNA obtained by fusion with corresponding cDNA according to techniques known in the art.

The term “derivative of the zyxin protein” means any polypeptide modified, for example, chemically by association with functional chemical groups, the functional chemical groups being selected from among the groups capable of realizing the coupling of the protein or a fragment thereof either with other molecules such as, for example, markers, of carrier proteins with the goal of fabricating an immunogen, enzymes, or solid supports, such as mineral supports or organic polymers.

A first mode of implementation pertains to a pharmaceutical composition comprising the zyxin protein or a functional fragment thereof.

According to this first mode of implementation, the pharmaceutical composition of the invention comprises the zyxin protein or functional fragments thereof associated with transport vectors selected from among the group comprising:

lipid systems such as anionic or neutral liposomes, and notably liposomes based on phosphatidylcholine (PC) or dioleylphosphatidyl choline (DPE), cationic liposomes, notably liposomes based on dioctadecyldimethyl ammonium bromide (DODAB), dioleyloxypropyl-trimethyla-mmonium (DOTMA), DOGS (Transfectam®), DDPPES, etc., cationic emulsions such as emulsions based on soy and 1,2 diolcoyl-glycero-3-trimethylammoni-um propane (DOTAP) and the like,

particular systems: as nonlimitative examples, either microparticles based on poly(lactide co-glycolide) acid (PLG), cetylmethylammonium bromide (PLG-CTAB), PLG-PEI, or microparticles based on PLG-poly-L-lysine, etc., or nanoparticles based on chitosan, nanoparticles of PLG, gelatin and the like,

polymer or polyplex systems based on poly-L-lysine, poly-ethylene-imine (PEI), dendrimer polyamidoamines, cationic polymers such as chitosan, DEAE-dextran, copolymers of TMAEM (trimethylammonium ethyl methacrylate) and N-2-hydroxypropyl methacrylamide (HPMA),

peptide systems such as the peptide RAWA, and

cationic polyene antibiotics such as the cationic derivatives of amphotericin B.

In the pharmaceutical composition of the invention, the zyxin is advantageously associated with intracellular transport vectors by covalent or noncovalent chemical bonds.

A second mode of implementation of the invention pertains to a pharmaceutical composition comprising as an active agent a nucleic acid molecule comprising a cDNA of the zyxin gene, a fragment or a derivative thereof.

According to this second mode of implementation, the pharmaceutical composition comprises a nucleic acid comprising a cDNA of the zyxin gene, a fragment or derivative thereof associated with a vector of viral recombinant expression or a vector of nonviral transport of particular type.

The term “association between the active agent and the transport vector” means attachment of the active agent to the transport vector by, for example, noncovalent bonds, for example, of hydrophobic type, or by covalent chemical bonds by means of coupling agents or not, by means of techniques well known in the art, either by insertion of the active compound in a viral or bacterial vector of recombinant expression. In this latter case, the active compound is brought to its target either by infection with viral particles expressing the active compound, or by transfection with vectors of recombinant expression that express the active compound during its integration in the host cells.

The pharmaceutical composition of the invention advantageously comprises a vector of recombinant viral expression comprising the elements required for transcriptional control as well as control of the translation of the cDNA sequence of the zyxin gene when the expression vector is introduced into target cells.

The pharmaceutical composition of the invention advantageously comprises a vector of recombinant viral expression comprising regulation sequences such as constitutive or inducible promoters, i.e., noncoding sequences of the zyxin gene, enabling the expression of zyxin in the cells of the host to which is administered the composition of the invention.

The pharmaceutical composition of the invention advantageously comprises a vector of recombinant viral expression comprising regulation sequences selected from among the LTRs sequences, such as, for example, the LTRs sequences of Moloney's leukemia virus, under the dependence of a 5′ LTR promoter.

The pharmaceutical composition of the invention preferably comprises as a vector of intracellular transport, any vector of recombinant viral expression placed under the control of the host cell allowing the expression of zyxin in the host cell according to the genetic recombination techniques well known in the art.

A nonlimitative example includes expression vectors stemming from adenovirus, recombinant adenovirus associated virus (AAV), recombinant baculovirus or retrovirus, and especially preferably a vector of recombinant lentivirus type.

In an especially preferred manner, the pharmaceutical composition of the invention comprises a vector of viral expression comprising sequences of promoters selected, for example, and in a nonlimitative manner, from among the promoter CMV, the promoter EF1 alpha or the promoter PGK.

According to a particular mode of implementation, the pharmaceutical composition of the invention comprises as an active agent a cell stemming from a patient with a tumoral pathology genetically modified for expressing the zyxin gene. The pharmaceutical composition of the invention is useful for the treatment or the prevention of tumoral pathologies. The pharmaceutical composition of the invention is useful for the treatment of pathologies such as the malignant hemopathies association with chromosomal anomalies of the localization region of the zyxin gene 7q34/q35. Thus, the pharmaceutical composition of the invention is useful for the treatment or the prevention of hepatocarcinomas, neuroectodermal cancer and Ewing's sarcoma.

The invention also pertains to the vectors of nonviral and viral intracellular transfer associated with the active agent used in the pharmaceutical composition as stated above.

The invention also pertains to a viral vector that can be used in a pharmaceutical composition as defined above. Thus, the invention includes a viral vector comprising a cDNA coding for the zyxin gene or a functional fragment thereof. More particularly, the viral vector is selected from among a recombinant vector stemming from an adenovirus, an adenovirus associated virus (AAV) or a retrovirus.

A third mode of implementation of the invention pertains to a pharmaceutical composition comprising as an active agent a cell characterized in that it is genetically modified to overexpress the zyxin gene or a functional fragment thereof. The overexpression of the zyxin gene by the cell is advantageously obtained either by transfection of a cell with expression vectors comprising a cDNA of the zyxin gene or by infection of a cell with viral particles expressing the zyxin gene.

The pharmaceutical composition of the invention preferably comprises as an active principle a cell selected from among a stem cell, a bone marrow cell, a hemopoietic cell or a hepatocarcinoma cell genetically modified to overexpress the zyxin gene or a functional fragment thereof. The pharmaceutical composition of the invention preferably comprises as active agent a CD34+ cell genetically modified to overexpress the zyxin gene or a functional fragment thereof. The pharmaceutical composition of the invention most preferentially comprises as active agent a cell stemming from a patient with a tumoral pathology genetically modified to express the zyxin gene or a functional fragment thereof.

The invention also includes a genetically modified cell overexpressing the zyxin gene and a genetically modified cell underexpressing the zyxin gene. Such a cell can be obtained, for example, by an antisense RNA targeting of the AUG of zyxin and introduced into the cells by the intermediary of a synthesis oligonucleotide cloned in the shuttle comprising the transport vector. The genetically modified cells according to the invention are preferably selected from among a stem cell, a bone marrow cell or a hepatocarcinoma cell. The genetically modified cells according to the invention are advantageously CD34+ cells.

According to a preferred mode of implementation, the genetically modified cells according to the invention are obtained from a patient with a tumoral pathology.

Another mode of implementation of the invention pertains to a pharmaceutical composition comprising as an active agent a compound binding polymerized actin F with an affinity constant greater by at least two logs than the affinity constant with which said active agent binds non-polymerized actin G. The active agents of the composition of the invention preferably have an affinity constant on the order of 10⁷-10⁸ M⁻¹ for polymerized actin F. According to a particular implementation of the invention, the active agent binding the polymerized actin is a cyclic peptide.

The invention also pertains to a nonhuman transgenic mammal comprising at least one genetically modified cell underexpressing the zyxin gene or a functional fragment thereof. The invention also includes a nonhuman transgenic mammal comprising at least one genetically modified cell underexpressing the zyxin gene or a functional fragment thereof. The invention also pertains to a method for the identification of compounds that stabilize the actin network of the cytoskeleton of a cell, including detecting a phenotypic reversion of the expression of zyxin induced by compound, characterized in that it comprises the following steps: bringing into contact the compounds to be tested with the cell, and quantification of the expression of zyxin in cell.

According to a particular implementation of the method of the invention, quantification of the expression of zyxin is effected by comparison of the expression of the zyxin messenger DNA in the cell in the presence or absence of the compound to be tested. According to another particular mode of implementation of the method of the invention, quantification of the expression of zyxin is effected by comparison of the expression of the zyxin protein in the cell in the presence and the absence of said compound to be tested.

The invention also includes method for the diagnosis of a tumoral pathology comprising the following steps: collecting cells from a patient, and quantification of the expression of zyxin in the collected cells. According to a preferred mode of implementation of the diagnostic method of the invention, quantification of the expression of zyxin is performed by measuring the expression of zyxin messenger RNA. According to another form of implementation of the diagnostic method according to the invention, quantification of the expression of zyxin is performed by comparison of the expression of the zyxin protein by cells collected at different intervals.

The invention also uses the method for the detection of a phenotypic reversion of expression of zyxin to determine the expression of an antitumor treatment in a patient by measuring the expression of the zyxin gene in the cells of the patient obtained at two different intervals during the antitumor treatment of the patient. Thus, the invention includes a method for the analysis of a tumor phenotype of a patient characterized in that it comprises the following steps: the collection of cells from the patient at two intervals of different times, quantification of the expression of zyxin in the cells collected at the different intervals, and comparison of the two levels of expression to constitute a differential phenotypic profile of the patient.

According to a mode of implementation of the method of analysis of the invention, quantification of the expression of zyxin is performed by comparison of the expression of the messenger RNA of the cells collected at different intervals. According to a mode of implementation of the method of analysis of the invention, the quantification of the expression of zyxin is performed by comparison of the expression of the zyxin protein by the cells collected at the different intervals.

The invention pertains to a method for the screening of an active compound in the treatment of cancers, comprising the following steps: incubation of tumor cells with the active compound, and measurement of the stabilization of the polymerization of the actin network of the cells.

The invention also pertains to the use of a substance capable of reestablishing the actin network of a cell for the preparation of a noncytotoxic antitumor drug.

The invention also uses such a substance for the treatment and/or prevention of a pathology resulting from a chromosomal anomaly at the level of the long arm of chromosome 7, more particularly at the level of region 7q34/q35. The invention also uses such a substance for the treatment of a malignant hemopathy associated with a chromosomal anomaly in the region 7q34/q35 of the zyxin gene.

The invention also uses a substance for the treatment or prevention of hepatocarcinomas or neuroectodermal cancers, the treatment or prevention of mesenchymal tumors, notably sarcomas, and the treatment or prevention of Ewing's sarcoma.

### Material and Methods

### Correlation Between Tumor Phenotype and Underexpression of Zyxin

### Description and Cell Cultures

A set of cell lines was cultured at 37° C. in a moist atmosphere containing 5% CO₂. With the exception of the GP+envAm12, they were maintained in DMEM medium (GIBCO) supplemented by 10% of fetal calf serum (GIBCO) and antibiotics (penicillin at 100 IU/ml and streptomycin at 100 μg/ml).

The EWS-FLI line contains a cDNA coding for the fusion protein EWS-FLI-1 in its genome. The expression of this protein is selected by means of 2.5 μg/ml of puromycin.

The line AS-A, developed by M. Hervy et al., produces a small antisense RNA directed against the mRNA coding for the protein EWS-Fli-1. This line is selected, in addition to puromycin, by 1 mg/ml of geneticin. Geneticin makes possible the selection of cells which produce the small antisense RNA directed against the mRNA coding for the protein EWS-FLI-1. The lines NIH3T3 AS zyxin are cells that produce a small antisense RNA directed against the AUG of mRNA coding for zyxin. They are cultured in a medium supplemented with geneticin at 1 mg/ml in order to select the expression of the antisense. The cells GP+env Am12 are transcomplementary cells capable of providing in trans the proteins coded by the genes gag and pol, carried on a plasmid, and env carried on another plasmid. This line is capable of producing amphotropic viral particles. This line is cultured in a medium containing DMEM and 10% of fetal calf serum (GIBCO) supplemented by penicillin and streptomycin. These cells are selected by means of a mixture of three compounds (200 μg/ml of hygromycin B, 15 μg/ml of hypoxanthine, 250 μg/ml of mycophenolic acid) over two weeks with transfection by the retroviral shuttle.

### Immunofluorescence

The cells were cultured on glass slides until they adhered (from 24 to 48 h). The cells were fixed with a 3% solution of paraformaldehyde rinsed with PBS and permeabilized with a PBS/0.2% triton X100 solution. The permeabilized cells were saturated with a PBS/2% BSA solution. In the case of immunostaining of the zyxin protein, the cells were incubated with the primary antibody (anti-zyxin of J. Wehland) diluted twice over 40 minutes, rinsed three times 5 minutes with PBS and then incubated with the secondary antibody murine anti-IgG coupled with Texas Red (TRITC) for 40 minutes. For the immunostaining of actin, the cells were incubated directly with phalloidin coupled with FITC for 40 minutes. The slides were observed with a fluorescence microscope.

### Construction

Generation of the pLNCX ADA (Adaptator) Vector from pLNCX

The retroviral vector pLNCX contains a part of the sequences LTRs and the sequence psi stemming from Moloney's murine leukemia virus (MoMLV) and in addition the neomycin resistance gene, conferring resistance to geneticin, under the dependence of the promoter of LTR in '5. This vector also contains a multicloning site (MCS) directly under the dependence of the early promoter of the cytomegalovirus (pCMV). The vector pLNCX is directed by HindIII/ClaI at the level of the MCS to insert there a sequence containing two adaptators which are capable of autoassociating in a complementary manner. Between these two adaptators, this sequence contains other unique restriction sites including NsiI and SalI.

### Generation of a Retroviral Vector Coding for Human Zyxin

The plasmid pzyxin GFP contains cDNA coding for human zyxin phase coupled with the gene of the green fluorescent protein GFP (provided by M. Beckerle). It is directed by Hind III and BamH I then cloned by the vector PLNCX at the level of the MCS (HindH I/BglII); BamHI and BglII are compatible sites. The digestion by Hind II/BglII eliminates one of the two adaptators, this preventing an autoassociation of the RNA coding for the zyxin produced by this vector.

The result of this construction is a retroviral vector named pLNCX ADA zyxin, coding for human zyxin under the direct influence of pCMV.

Generation of a Vector Producing an Antisense Directed Against Zyxin: pLNCX ADA as Zyxin

pLNCX ADA as zyxin is a vector which has the capacity of producing a small RNA in loop rod structure directed against the AUG of the mRNA coding for zyxin, directly under the dependence of the promoter pCMV.

The construction of the vector is implemented by inserted at the level of the sites NsiI and Sal I of the MCS a small sequence directed against the AUG of the mRNA of zyxin.

### Transfection

The retroviral shuttle of the vector is transformed into the corresponding virus by transfecting the retroviral in a transcomplementary cell line GP+envAM12 (GPA). The transcomplementary GPA line is transfected by the retroviral shuttle (PLNCX zyxin or PLNCX ADA as zyxin) in the presence of Superfect (Qiagen) according to the recommendations of the supplier.

The cells expressing the gene neo^{r} are selected in a medium containing 1 μg/ml of G418. The resistance cells are collected, amplified and cultured in 75-cm² flasks at the rate of 2·10⁶ cells. Two days later, the medium is replaced by an unselective medium. The supernatant is collected every 24 hours for 3 days, regrouped, broken down into aliquot portions and frozen. The retroviral titer is evaluated on the NIH3T3 cells after selection of the cells with geneticin (1 mg/ml). The viral supernatant of the GPA is then used for infecting the desired cells with a multiplicity of infection of the order of 0.1. Three days after the infection, the cells were selected with 1 μg/ml of G418. Only the cells that had integrated the retroviral shuttle resistant to G418 and formed clones were isolated and amplified so as to produce permanent cell lines.

### Immunoprecipitation

The adherent cells, were trypsinated, residualized and rinsed with PBS. The cells were lysed with cold RIPA (10 mM Tris-HCl pH 7.4, 100 mM NaCl, 1 mM EDTA, 1% Triton X100, 0.5% Na deoxycholate, 0.1% SDS) in the presence of a protease mixture (Boehringer). After 20 minutes of incubation on wheel at 4° C., the samples were centrifuged for 15 minutes at 14,000 rpm. The supernatants were recovered and the protein concentration was determined by Bradford's test. An aliquot containing 1.5 mg of total protein extract was incubated for 1.5 hours at 4 hours with 0.2 μg of antibody directed against the C terminal domain of Fli-1 (Santa Cruz SC-356) and then for 1 hour with 20 μl of a mixture of agarose beads coupled with protein G (Sigma). After three washings in cold RIPA, the beads were resuspended in 20 μl of the buffer Laemmli 2× and then brought to boiling for 10 minutes. The samples were then analyzed by a classic immunoblot using for incubation of the primary antibody, a solution containing 1 μg of anti-Fli-1 in 5 ml of TBS-0.1% (v/v) Tween).

### Western Blot

Analysis of the zyxin protein production level was performed by Western blot on a total cell extract using as primary antibody the murine monoclonal anti-zyxin antibody provided by J. Wehland directed against the region located between the NEW and the LIM domains. This membrane was developed by a chemiluminescent reagent (Immunostar: Biorad).

### RNA Analysis

### Northern Blot

Extraction of the total RNA of the different cells lines was performed using the lyse solution RNAplus (Quantum Biotechnologie) in accordance with the supplier's instructions. An aliquot of 10.μg of total RNA was denatured in a solution containing 0.04 M MOPS pH 7, 0.01 M of sodium acetate, 2.2 M of formaldehyde and 50% formamide. The samples were analyzed on formaldehyde-denatured agarose gel and transformed onto a charged nylon membrane (Hybond N+: Amersham Pharmacia). The membrane was hybridized with a cDNA probe radio-labeled with [³³P] dCTP by random priming (Prime-a-gene® labeling system: Promega) in a prehybridization system containing 5×SSC, 5×Denhardt's, 0.1 mg/ml of salmon sperm DNA, 0.1 mg/ml of yeast t-RNA, 0.1% SDS, 25 mM pH 7 KH₂PO₄ and 50% formamide at 42° C. overnight. The next day the membrane was rinsed three times with SSC2×/0.1% SDS at ambient temperature and once to twice at 42° C. with 0.5×SSC/0.1% SDS. The signals of the membrane were then observed with the phosphoImager.

### RT-PCR

The total RNAs were produced by the same technique as described above. The quality and cleanliness of the RNAs were verified on denaturing gel. The reverse transcription was performed using the Qiagen Omniscript™ kit in a specific manner with a 20 mers LTR primer. The conditions used were 10 ng of LTR primer, 2.5 mM of dNTP, 1 μg of total RNA supplemented by 2 OU of RNase inhibitor (RNAsin®), buffer RT and 4 OU of reverse transcriptase. The mixture was incubated for one hour at 37° C. and for 5 minutes at 94° C. The RT products of the specific cDNAs were amplified by PCR using two other primers named as 1 and as 2. The reaction conditions were 0.25 mM dNTP, 100 ng of each of the primers and 1/20^{th} of the RT product, 1.5 mM MgCl₂, buffer Taq pol and 1 U of enzyme Taq pol (Perkins Elmer N801-0060). The cycles employed were 4 minutes 94° C. and 30 cycles (30 s 94° C., 45 s 61° C., 1 min 72° C.) and 10 minutes at 72° C.

| | |
|---|---|
| LTR: AGATATCCTGTTTGGCCAT | (SEQ ID NO: 1) |
| AS1: GCCGTGCATCATCCTGACTG | (SEQ ID NO: 2) |
| AS2: CTGTTCCTGACCTTGATCTG | (SEQ ID NO: 3) |

### Tumorigenicity Test

Cells obtained from different clones to be tested were trypsinated, residualized and resuspended in sterile PBS at the rate of 5·10⁶ cells per ml. A 200-μl aliquot of cells was injected via the subcutaneous route in nude mice aged 6 to 8 weeks, irradiated the previous evening at 5 Gray. The mice were raised in a sterile, climate-controlled atmosphere. Observation of the development of tumors was performed weekly for 5 to 6 weeks.

### Macroarray Experiment

The cDNA expression card “Atlas cDNA expression Arrays” (Clontech) was implemented according to the supplier's instructions. Two identical nylon membranes (no. 7741-1) on which were deposited 588 samples of murine cDNA, corresponding to 588 genes, made possible parallel hybridization of the cDNAs of two different cell lines. Information regarding the 588 genes is available in the Clontech catalogue. The preparation of the radiolabeled cDNA probes was implemented by reverse transcription with [³²P] dATP using the Clontech kit. Hybridization was performed according to the supplier's instructions. The signals were observed with the PhosphoImager.

### RESULTS

### Role of Zyxin in the Cellular Transformation Induced by the Fusion Protein EWS-FLI.

### Establishment of EWS-FLI Lines Overexpressing Zyxin

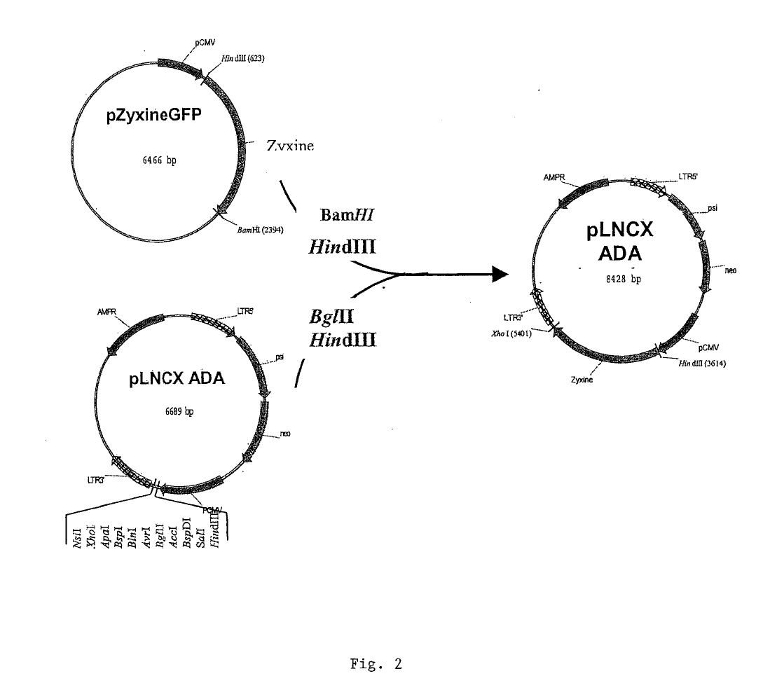

Overexpression of zyxin in NIH3T3 cells expressing the fusion protein EWS-Fli (EWS-FLI) was achieved by infection using the retroviral shuttle pLNCX. The open reading frame corresponding to zyxin, stemming from the plasmid pZyxin-GFP (FIG. 2) was introduced into the multicloning site of the plasmid pLNCX located downstream of the promoter CMV. There was thereby obtained the plasmid named pLNCX-zyxin which contains both LTR sequences (5′ and 3′), the sequence PSI+ necessary for the encapsidation of the retroviral RNA, the gene Néo^{R} responsible for resistance to geneticin under the dependence of LTR 5′ and the reading frame of human zyxin under the influence of the promoter CMV (FIG. 2).

The production of virus was obtained by transfection of this plasmid in a amphotropic murine encapsidation line named GPA. The EWS-FLI cells were infected by means of the viral supernatant produced by the GPA cells and selected in the presence of geneticin.

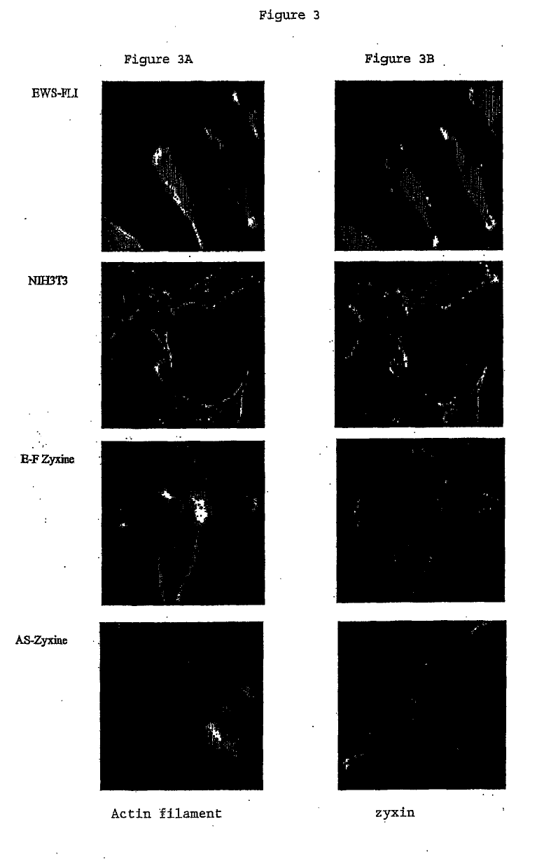

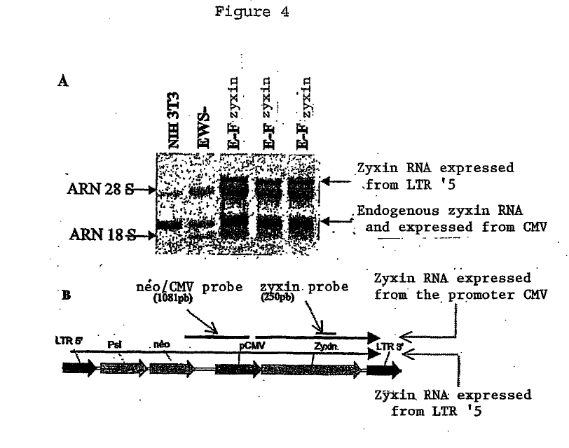

The clones obtained in this manner were named E-F/Zyxin. Fluorescence microscopy study (FIG. 3) showed that the EWS-FLI lost their actin microfilament bundles and the capacity to expand, characteristics typical of transformed fibroblasts (Pollack, R. et al; 1975, Maness, P. et al; 1982). In contrast, the cells of the E-F/Zyxin clones had partially recovered the structure of the actin microfilaments as well as the expansion capacity of the NIH3T3 cells (FIG. 4). Moreover, these cells no longer had the capacity of growth in multilayers, typical of transformed cells. In parallel with these structural modifications, we observed a relocalization of zyxin at the level of the adherence plaques, in the intercellular junctions and along the stress cables (FIG. 3). Expression of zyxin mRNA in E-F zyxin cells.

Northern blot analysis showed that the RNA coding for zyxin is more weakly expressed in the tumorigenic EWS-FLI line than in the NIH3T3 line (FIG. 4). This result confirms the difference in expression observed previously, by microarrays, between the NIH3T3 and EWS-FLI cells. The anticipated size of the human zyxin mRNA stemming from the retroviral shuttle and expressed from the promoter CMV (2.2 kb) (FIG. 4B) is identical to that of the endogenous mRNA of zyxin. It is thus very probable that the increase in the intensity of the band that is seen in the three E-F/zyxin clones is due to the expression of the zyxin RNA expressed from the retroviral shuttle. Moreover another RNA of larger size at 4.7 kb is represented solely in the RNAs stemming from the E-F/zyxin clones. This RNA has a size comprised between 5.5 and 6 kb compatible with an RNA that would be expressed from the promoter located in the U3 region of the LTR 5′ (5.7 kb) of the retroviral shuttle (FIG. 4B).

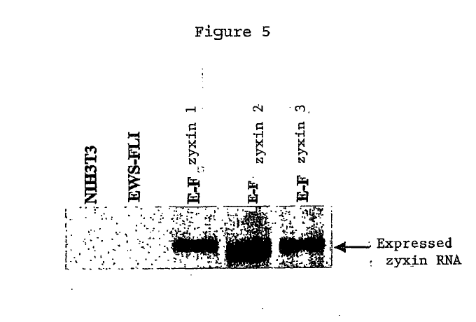

To verify this hypothesis, a second Northern blot was performed using a probe (neo/CMV) of 1081 pb, corresponding to a restriction fragment stemming from the plasmid pLNCX ADA zyxin, capable of detecting solely RNA stemming from the LTR 5′ (FIG. 5).

The results presented in FIG. 5 reveal hybridization solely in the E-F zyxin clones. Referring to the position of the RNA 28S, observable under UV, the detected band is positioned at the same level as the undetermined band present in the Northern blot using the zyxin probe. We can therefore conclude that the retroviral shuttle produces two RNAs containing the zyxin sequence, one stemming from the promoter CMV and other from the promoter present in the LTR 5′.

### Overexpression of Exogenous Zyxin Protein.

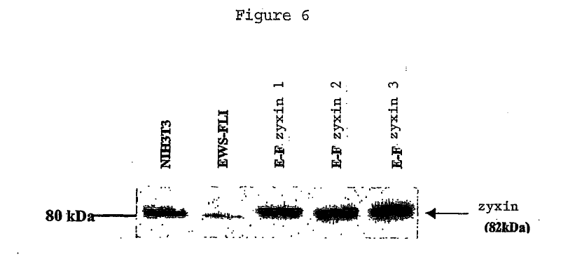

To determine whether the E-F zyxin clones containing zyxin RNA stemming from the retroviral shuttle are capable of producing the corresponding protein, a Western blot immunodeveloped by anti-zyxin antibody was implemented. The results of this experiment are presented in FIG. 6. For the set of lines, we detected a specific and unique band of a protein of molecular mass slightly greater than 80 kDa. This molecular mass is in agreement with the apparent molecular mass of zyxin (82 kDa) reported by Schmeichel et al., 1998. The underexpression of zyxin mRNA in the EWS-Fli compared to the NIH3T3 line is manifested by a decrease in the corresponding protein.

Similarly, the overexpression of zyxin at the RNA level of the E-F zyxin 1, 2 and 3 is manifested by a restoration of the level of zyxin protein close to that of the NIH3T3 line. These results thus show a correlation between the level of RNA produced by the cells and the level of protein expressed. In conclusion, the introduction of cDNA coding for zyxin in transformed EWS-FLI enables restoration of the level of expression of this protein to a level that is comparable or even greater than that of the parent NIH3T3 cells. The overexpression of zyxin protein is very probably due to the RNA expressed from the CMV promoter. In fact, in the case of minority RNA stemming from the LTR 5′, the reading frame of the phosphotransferase APH (3′) II (Davies and Smith, 1978) conferring geneticin resistance on the cells is translated. The absence of internal translation initiation sequence thus prevents the open reading frame of human zyxin downstream to be translated.

### Expression of EWS-FLI-1 in E-F Zyxin Clones.

In parallel it was verified that E-F zyxin clones studied conserve the expression of EWS-FLI-1 protein, responsible for the tumorigenic character.

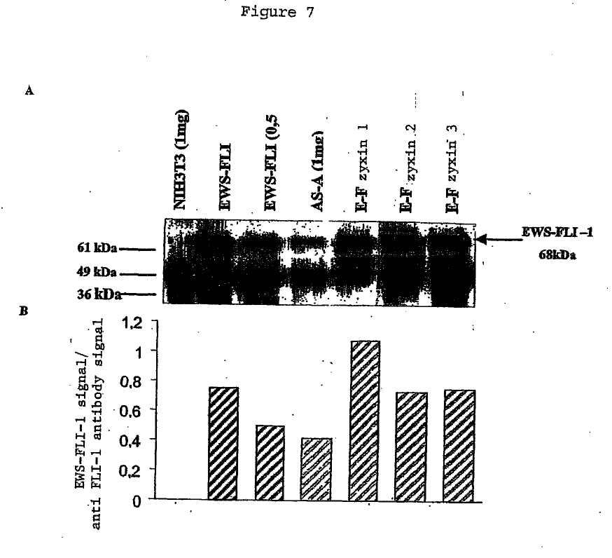

To do this, a study of the expression of EWS-FLI-1 protein was performed by Western blot after immunoprecipitation of the protein extracts from the E-F zyxin clones (FIG. 7). The results, presented in FIG. 7, show a specific detection of a protein of molecular mass greater than 61 kDa. This mass is compatible with the expected apparent molecular mass of EWS-FLI-1 protein (68 kDa). Other bands appear under this band. They correspond to the denaturation product of the antibody used for the immunoprecipitation (heavy chains (50 kDa) of anti-Fli-1 antibody).

The difference in intensity detected in the presence of two different quantities of protein extract show that the amount of antibody used is not limiting. To be able to compare the intensity of the bands corresponding to the EWS-FLI-1 protein, the amount of EWS-FLI-1 immunodetected by the amount of anti-Fli-1 antibody detected was corrected. The results presented in histogram form (FIG. 7B) indicate the absence of EWS-FLI-1 protein in the NIH3T3 line and an underexpression in the AS-A line (expressing an antisense directed against the EWS-FLI junction sequence) compared to the EWS-FLI line. For the E-F zyxin clones, the quantity of EWS-FLI-1 protein was clearly greater than the quantity of protein detected in the non-tumorigenic AS-A line and remained comparable to the quantity present in the tumorigenic EWS-FLI cells.

We can assume that these cells conserve a sufficient quantity of EWS-FLI-1 protein to induce subcutaneous tumors in nude mice. Thus, the possible loss of tumorigenicity of the E-F zyxin clones would not be due to a decrease in the expression of EWS-FLI-1 protein.

### Tumorigenicity of the E-F Zyxin Clones

Determination of the induction of a loss of malignant phenotype in the nude mouse by the overexpression of zyxin in the EWS-FLI line is illustrated in Table 1 below.

TABLE 1
Development of tumors in the nude mouse.

| | Elapsed time after the injection | | | | |
|---|---|---|---|---|---|
| | 1 | 2 | 3 | 4 | 5 |
| | week | weeks | weeks | weeks | weeks |
| NIH3T3 | 0/5 | 0/5 | 0/5 | 0/5 | 0/5 |
| EWS-FLI | 0/4 | 1/4 | 4/4 | 4/4 | 4/4 |
| AS-A | 0/4 | 0/4 | 0/4 | 0/4 | 1/4 |
| E-F zyxin 1 | 0/5 | 0/5 | 0/5 | 0/5 | 2/5 |
| E-F zyxin 3 | 0/5 | 0/5 | 0/5 | 1/5 | 2/5 |

These results correspond to the study of the number of tumors developed in the nude mouse after subcutaneous injection of 10⁶ cells of different cell lines over 5 weeks. The cells were injected 24 hours after irradiation of the mice at 5 Gray.

The injection of NIH3T3 cells did not lead to the development of tumors. This line is used as a negative control because it is known to be non-tumorigenic. In contrast, for the line EWS-FLI, known to be tumorigenic, all of the mice developed tumors between the second and third weeks. With regard to the tumorigenicity study of the E-F zyxin clone, two scenarios can take place: either no development of tumors takes place (three out of five mice) or there is a delay in the development of tumors of about two to three weeks (two out of five mice). Analysis of these tumors showed that the DNA of the retroviral shuttles is always present; in contrast, exogenous RNA coding for zyxin was not detected. It would thus appear that the delayed development of tumors in the mice was due to a loss of expression of exogenous zyxin protein. Underexpression of zyxin in the acquisition of the tumor phenotype.

### Establishment of NIH3T3 Lines Underexpressing Zyxin.

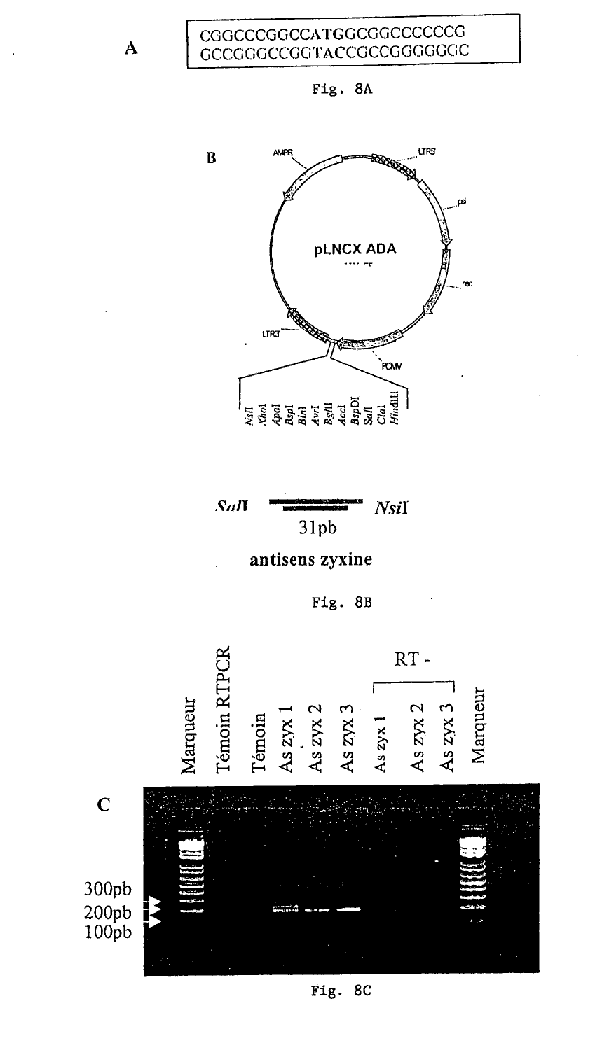

Given the importance of the level of expression of zyxin in the maintenance of the tumor phenotype of NIH3T3 cells transformed by EWS-FLI fusion protein, we determined the consequences of a forced diminution of this protein in non-tumorigenic cell lines. To do this, a small antisense RNA targeting zyxin AUG was used (FIG. 8A). This antisense RNA was introduced into the cells by the intermediary of a synthesis oligonucleotide cloned in the pLNCX shuttle at the level of the NsiI/SalI restriction sites (FIG. 8B).

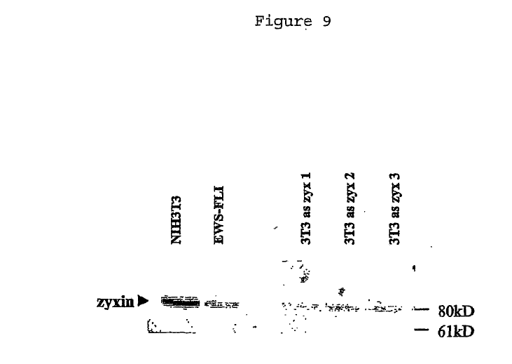

In the three clones selected (in the presence of G418 1 mg/ml) and which were named AS-ZYX 1, 2 and 3, the expression of the antisense (FIG. 8C) was accompanied by a diminution of zyxin protein (FIG. 9). This diminution of expression was on the same order as that detected in the NIH3T3 cells transformed by EWS-FLI fusion protein.

This diminution in the level of expression of zyxin in NIH3T3 cells was manifested in a noteworthy morphological change in the cells. Thus, we observed a noteworthy loss of cytoplasmic expansions and adherence as well as noteworthy changes in the structure of the actin filaments (FIG. 3). These morphological changes, typical of transformed cells, were also accompanied by modifications in the multiplication characteristics of the cells. Thus, the doubling time of these cells (20-22 hours) was intermediate between that of the transformed EWS-FLI cells (17-18 hours) and that of the NIH3T3 parent cells (24-26 hours). These data also indicate that the AS-zyxin cells, like the EWS-FLI cells, had loss contact inhibition.

### Tumorigenicity of the AS-Zyxin Clones

The tumor-development tests in the nude mouse confirmed the morphological changes and growth modifications observed between the cells expressing the antisense directed against zyxin and the parent NIH3T3 cells (Table 2). These is a slight delay in the appearance of tumors from the AS-zyxin clones compared to that observed from the EWS-FLI tumoral line, but the three clones did develop tumors. The development rate of the tumors after injection of EWS-FLI cells is also more rapid than that seen after injection of the AS-zyxin clones. These two observations would appear to stem from the intrinsic multiplication rate of the cells.

TABLE 2
Table 2: Tumorigenicity test: study of the number of tumors developed
after subcutaneous injection in the nude mouse of 10⁶ cells of
different cells lines over weeks.
Comparison of the profile of expression of genes between the line
NIH3T3 and AS-zyxin.
Development of tumors in the nude mouse.

| Line | 1 | 2 | 3 | 4 | 5 | 6 |
|---|---|---|---|---|---|---|
| NIH3T3 | 0/4 | 0/4 | 0/4 | 0/4 | 0/4 | 0/4 |
| EWS-FLI | 0/4 | 0/4 | 4/4 | 4/4 | 4/4 | 4/4 |
| AS zyx 1 | 0/4 | 0/4 | 0/4 | 0/4 | 2/4 | 3/4 |
| AS zyx 2 | 0/4 | 0/4 | 0/4 | 1/4 | 2/4 | 4/4 |
| AS zyx 3 | 0/4 | 0/4 | 1/4 | 1/4 | 2/4 | 3/4 |

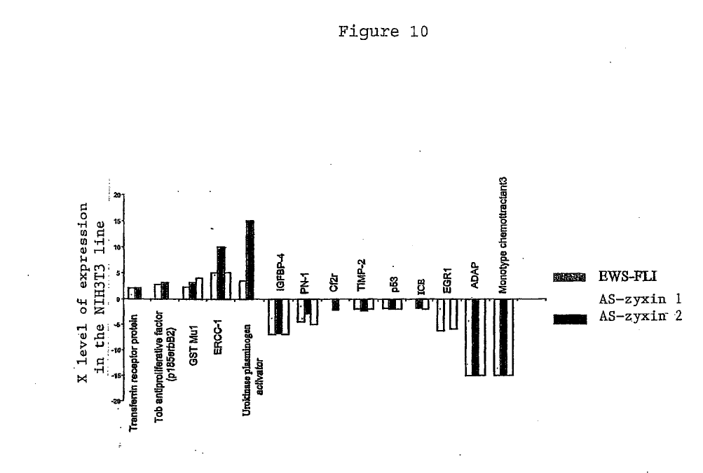

The comparative study of the expression of genes by the “cDNA expression arrays” technique between the NIH3T3 cells and the AS-zyxin 1 or 2 clones showed that the inhibition of zyxin disturbs genetic expression. Ten and thirteen genes respectively were identified in the AS-zyxin 1 and 2 clones whose expression was modified in relation to the NIH3T3 cells (FIG. 10). Nine among these different genes were common to the two clones.

These genes can be grouped into four families: the tumor suppressors (EGR1 and p53), the proteins involved in repair (ERCC-1), the proteins playing a role in differentiation and cell growth (ADAP, IGFBP-4, ICE) and the proteins intervening in the cellular matrix (TIMP2, PN-1 and urokinase plasminogen activator). Moreover, nine of these genes were identified in the analysis of the profiles of expression between the NIH3T3 parent line and the line transformed by EWS-FLI (FIG. 10). These results show very clearly that the level of expression of zyxin in the cells used influences the genetic regulation process.

### Pharmacological Approach of Treatment of Cancers by Stabilization of the Actin Network.

### In Vitro Measurement of the Polymerization of Actin by Fluorescence Anisotropy

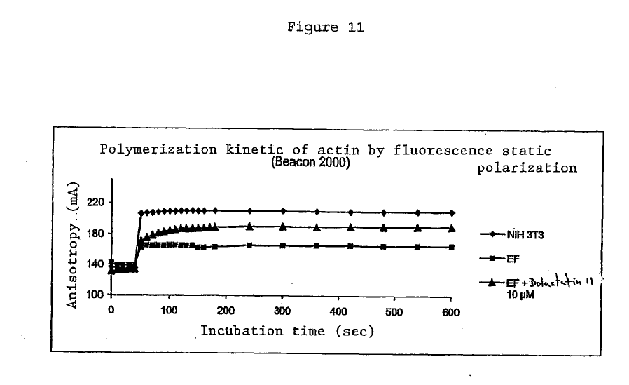

Cell extracts of NIH3T3 cells or EWS-Fli cells were brought into the presence of actin Alexa 488 in a buffer G (4.3 μM Tris pH 8.1; 170 μM CaCl₂; 170 μM DTT; 170 μM ATP). The polymerization reaction was triggered by addition of a buffer P (KCl 51 mM, MgCl₂ 1 mM and ATP 0.5 mM). Dolastatin (μM) was added to the cell extracts of the EWS-Fli cells at the same time as the buffer P. Polymerization of actin was monitored by fluorescence anisotropy on a Beacon 2000. The results obtained are illustrated in FIG. 11.

Morphological modification of the NIH3T3 and EWS-Fli cells in the presence of dolastatin (D11).

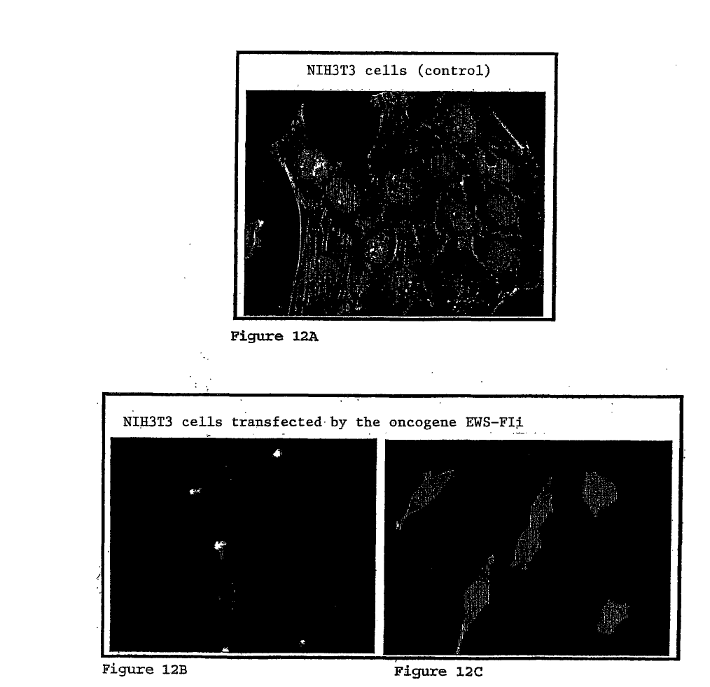

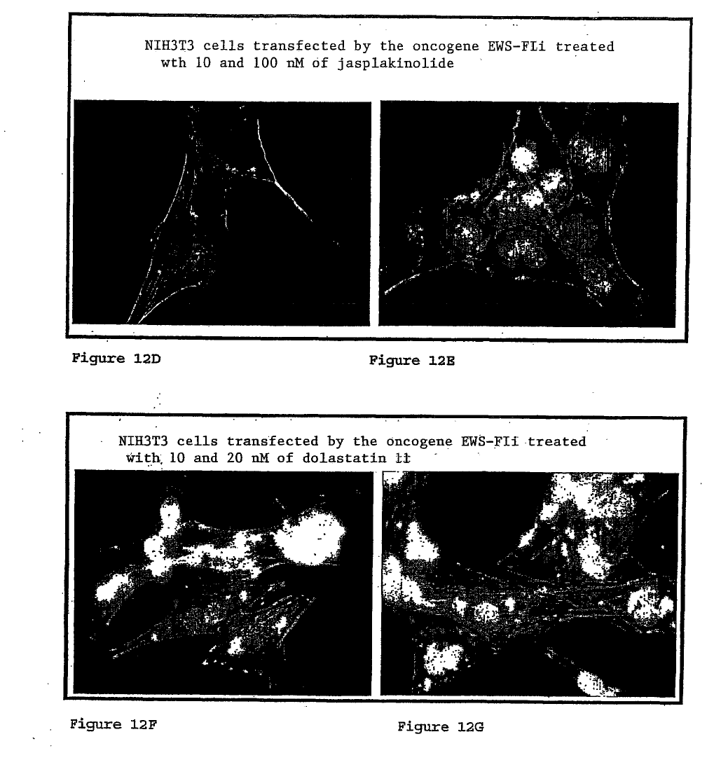

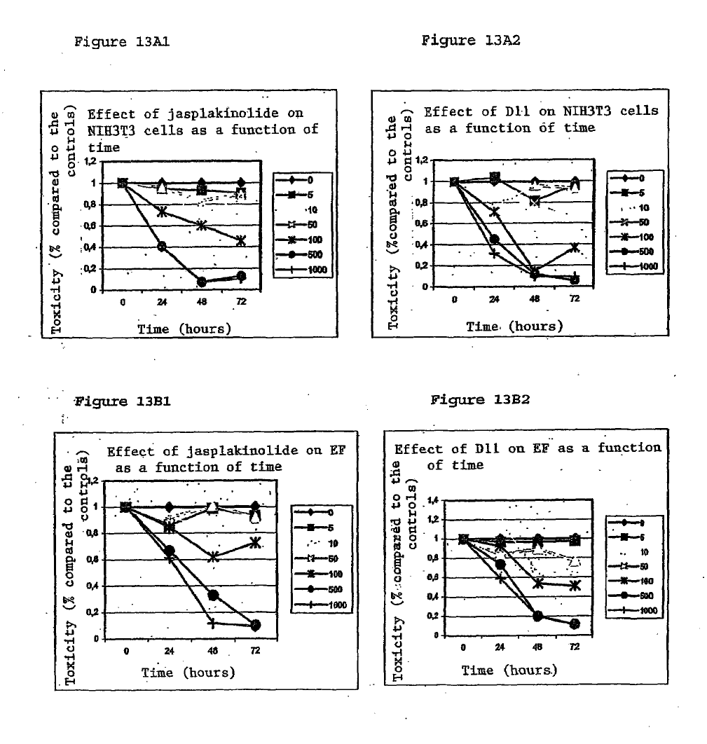

The cells (5×10³) were cultured on glass slides 10 mm in diameter and kept in culture for 24 hours. After fixation (3% paraformaldehyde) and permeabilization (0.2% TritonX100), the actin filaments of the cells were immunostained by phalloidin coupled to FITC. Untreated NIH3T3; NIH3T3 transfected by EWS-Fli (EF); EF incubated in the presence of 10 μM or 100 μM of jasplakinolide; EF incubated with 10 μM or 20 μM of D11 (FIG. 12).

These results show the effect of these two active agents, jasplakinolide and dolastatin 11, on the cytoskeleton.

### Toxicity of Dolastatin-11 (D11) on NIH3T3 and EWS-Fli Cells

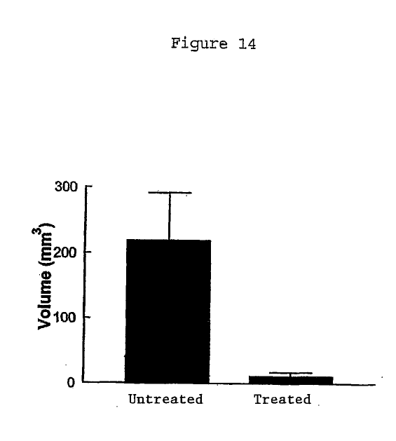

The cells (10⁴ per well) were cultured in 96-well plates in the presence of different concentrations of D11. Monitoring of the toxicity of D11 in relation to time was performed by means of an MTT test on the NIH3T3 cells (A) and the EWS-Fli cells (B). The results are illustrated in FIG. 13.

### Development of Tumors in the Nude Mouse in the Presence of Dolastatin-11.

Twenty-four hours after irradiation (5 Gy) of the mice, the EWS-Fli cells (10⁶) were inoculated via the subcutaneous route. The mice were then separated into two lots, with the first lot corresponding to the untreated mice and the second lot to the mice having received three injections of dolastatin 11 via the intravenous route (10 μg/kg) on the 5^{th}, 8^{th} and 11^{th} days after injection of the EWS-Fli cells. Measurements of the development of the tumors were performed on the 28^{th} day and are shown in FIG. 14. Displacement rate of the cells by phase-contrast microscopy.

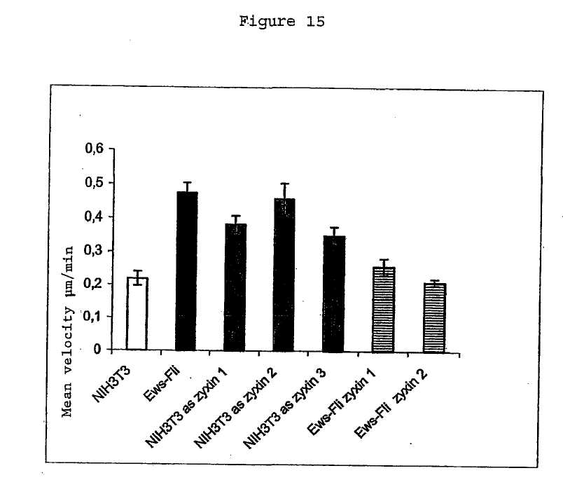

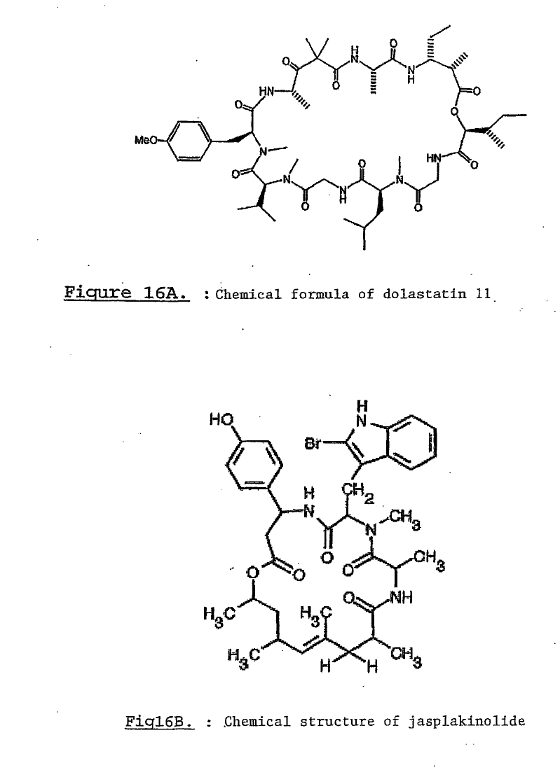

The cells were cultured (20% of confluence) on glass slides 20 mm in diameter. Three days after culturing, the glass slides were placed in hermetic chambers, with controlled temperature (37° C.) and CO₂ (5%). Measurement of the cell motility was performed by phase-contrast microscopy photography every 4 minutes for 24 hours. The motility analyses, performed using the program “metamorph”, were performed on ten cells for each cell type. The results are illustrated in FIG. 15.

### Analysis

The actin polymerization studies, using fluorescence anisotropy (FIG. 11), performed by the applicant on extracts of nontumoral cells (NIH3T3) and extracts of EWS-Fli tumoral cells show that the extracts of NIH3T3 cells have a much greater capacity to polymerize actin than the extracts of EWS-Fli cells. Nevertheless, the addition of dolastatin 11 (R Bai et al.; Molecular Pharmacology: 2001) in the polymerization buffer of the EWS-Fli cell extracts can compensate in part for this deficit of the EWS-Fli cells (FIG. 11).

In relation to these results obtained on the extracts, we analyzed whether this capacity of dolastatin 11 (D11) to stabilize polymerized actin could lead to a morphological reversion identical to that which was observed when the level of expression of zyxin protein was reestablished in these EWS-Fli tumoral cells.

Treatment of EWS-Fli cells with 10 or 20 nM of dolastatin 11 not only restored the stress fibers of the cells with a morphology close to that of NIH3T3 cells but also reestablished the contact structures between the cells (FIG. 12).

In contrast, the same concentrations of dolastatin 11 did not modify in the least the morphology of NIH3T3 cells (FIG. 12). In a completely surprising manner, for these low concentration of dolastatin 11, no cytotoxicity or cytostaticity effects were observed on either of the two cell lines (FIG. 13). The experiments performed on nude mice confirm the results obtained in vitro. The development of tumors due to the subcutaneous injection of EWS-Fli cells was markedly delayed by the intravenous injection of dolastatin 11 (FIG. 14). These experiments are especially most interesting because they were performed with a concentration of dolastatin 11 (10 μg/kg) five times weaker than the dose not affecting survival of the mice.

Although other compounds of the dolastatin family, such as D10 and D15 which target tubulin filaments, have been the object of multiple studies in which they were used as active compounds for antitumor treatments, they were used as cytotoxic agents intended to kill the tumor cell. The doses required for this approach lead to numerous undesirable side effects.

In contrast, the pharmaceutical compositions of the invention require for the phenotypic reversion of the tumor phenotype doses that are markedly lower than those known in the prior art.

These overall results show that it is possible to use a non-cytotoxic pharmacological approach to reestablish the actin cytoskeleton of tumor cells in the context of tumors induced notably by underexpression of zyxin.

One of the cancer families susceptible of being treated by the pharmaceutical compositions of the invention is the family of melanomas in which the applicant characterized the underexpression of the zyxin gene.

To obtain these results, we characterized the expression of multiple genes in a murine melanoma line in comparison to the expression of the same genes in a nontumoral line.

B16/F10 cells are a very aggressive murine melanoma line. They were compared to the non-tumorigenic NIH3T3 line.

The total RNAs of the two cell lines were amplified and radiolabeled by RT PCR. The RT PCR products (cDNA) were incubated with Atlas® Mouse cDNA Expression Array Clontech membranes (ref. 7741-1). The membranes were then developed and the images were analyzed with the MicroArray® computer program.

The results of this characterization are illustrated in Table 3 below.

TABLE 3

| | | | | |
|---|---|---|---|---|
| | | | Differ- | |
| Intensity 1 | Intensity 2 | Ratio | ence | Protein/gene |

Genes overexpressed in B16/F10 cells compared to NIH3T3 cells.

| | | | | | |
|---|---|---|---|---|---|
| 13734 | 22369 | 1,629 | 8635 | EB1 APC- | US1196 |
| | | | | binding protein | |
| 8952 | 31449 | 3,513 | 22497 | Met proto- | Y00671 |
| | | | | oncogene | |
| 17438 | 34520 | 1,980 | 17082 | G2/mitotic- | X64713 |
| | | | | specific cyclin | |
| | | | | B1 (CCNB1; | |
| | | | | CYCB1); CCN2 | |
| 7822 | 21439 | 2,741 | 13617 | G2/M-specific | X66032 |
| | | | | cyclin B2 | |
| | | | | (CCNB2; | |
| | | | | CYCB2) | |
| 18348 | 34001 | 1,853 | 15653 | cyclin D2 (G1/S- | M83749 |
| | | | | specific) | |
| 15532 | 32903 | 2,118 | 17371 | 84-kDa heat | M36829 |
| | | | | shock protein | |
| | | | | (HSP84); HSP | |
| | | | | 90-beta; tumor- | |
| | | | | specific | |
| | | | | transplantation | |
| | | | | 84-kDa antigen | |
| | | | | (TSTA); HSPCB | |
| 31460 | 52671 | 1,674 | 21211 | HSP86; heat | M36830 |
| | | | | shock 86-kDa | |
| | | | | protein | |
| 19970 | 34146 | 1,710 | 14176 | Erp72 | J05186 |
| | | | | endoplasmic | |
| | | | | reticulum stress | |
| | | | | protein; protein | |
| | | | | disulfide | |
| | | | | isomerase- | |
| | | | | related protein | |
| 16542 | 32910 | 1,989 | 16368 | 58-kDa inhibitor | U28423 |
| | | | | of RNA- | |
| | | | | activated protein | |
| | | | | kinase | |
| 16032 | 50452 | 3,147 | 34420 | glutathione S- | D30687 |
| | | | | transferase Pi 1 | |
| | | | | (GSTPIB); GST | |
| | | | | YF-YF | |
| 28766 | 41408 | 1,439 | 12642 | rac alpha | M94335 |
| | | | | serine/threonine | |
| | | | | kinase (RAC- | |
| | | | | PK-alpha); C-akt | |
| | | | | proto-oncogene; | |
| | | | | protein kinase B | |
| | | | | (PKB) | |
| 23556 | 52671 | 2,236 | 29115 | epidermal | L21671 |
| | | | | growth factor | |
| | | | | receptor kinase | |
| | | | | substrate EPS8 | |
| 7642 | 20421 | 2,672 | 12779 | non-histone | X53476 |
| | | | | chromosomal | |
| | | | | protein HMG-14 | |
| 14928 | 25099 | 1,681 | 12987 | granulocyte- | M85078 |
| | | | | macrophage | |
| | | | | colony- | |
| | | | | stimulating | |
| | | | | factor receptor | |
| | | | | low-affinity | |
| | | | | subunit precursor | |
| | | | | (GM-CSF-R) | |
| 8434 | 20475 | 2,428 | 12041 | CD44 antigen | M27129 |
| | | | | precursor; | |
| | | | | phagocytic | |
| | | | | glycoprotein I | |
| | | | | (PGP1); HUTCH | |
| | | | | I; extracellular | |
| | | | | matrix receptor | |
| | | | | III (ECMR III); | |
| | | | | gp90 lymphocyte | |
| | | | | homing/adhesion | |
| | | | | receptor; hermes | |
| | | | | antigen; | |
| | | | | hyaluronate | |
| | | | | receptor; LY-24 | |
| 31064 | 49983 | 1,609 | 18919 | phospholipase | D78647 |
| | | | | A2 | |

Genes underexpressed in B16/F10 cells compared to NIH3T3 cells.

| | | | | | |
|---|---|---|---|---|---|
| 23216 | 14134 | 0.609 | −9082 | zyxin (ZYX) | X99063 |
| 59496 | 1863 | 0.031 | −57633 | delta-like protein | L12721 |
| | | | | precursor | |
| | | | | (DLK); | |
| | | | | preadipocyte | |
| | | | | factor 1 | |
| | | | | (PREF1); | |
| | | | | adipocyte | |
| | | | | differentiation | |
| | | | | inhibitor protein; | |
| | | | | SCP-1 | |
| 45736 | 19192 | 0.420 | −26544 | early growth | M20157 |
| | | | | response protein | |
| | | | | 1 (EGR1); | |
| | | | | KROX-24 | |
| | | | | protein; ZIF/268 | |

Genes overexpressed in B16/F10 cells compared to NIH3T3 cells.

| | | | | | |
|---|---|---|---|---|---|
| 55682 | 6916 | 0.124 | −48766 | insulin-like | X81584 |
| | | | | growth factor | |
| | | | | binding protein- | |
| | | | | 6 (IGFBP 6) | |
| 31382 | 8562 | 0.273 | −22820 | protease nexin 1 | X70296 |
| | | | | (PN-1) | |
| 56386 | 27327 | 0.485 | −29059 | c-myc proto- | X01023 |
| | | | | oncogene | |

These results show that the zyxin gene is underexpressed in the melanomas.

Our experiments were able to establish a relation between the level of zyxin expression in cells and the acquisition or the maintenance of the tumor phenotype. The study of the role of zyxin in neoplastic transformation was performed subsequent to multiple indirect observations:

A) The acquisition of the tumor phenotype by NIH3T3 cells following the expression of EWS-FLI fusion protein in these cells, as well as the loss of tumorigenicity, due to the extinction of this oncogenous protein, accompanied by noteworthy morphological modifications (FIG. 3).

B) The fact that the malignant transformation is generally manifested by modification of the adherence and motility capacities. These modifications are always linked to a destruction of the actin filaments.

C) Among the ten genes identified as being directly under the dependence of the oncogenous EWS-FLI protein, zyxin is the only one that plays a role in the formation of the structure of the cytoskeleton, adherence and cellular motility.

To establish a direct link between zyxin and malignant transformation, on the one hand, a vector enabling reestablishment of the expression of zyxin was introduced into a tumoral line (EWS-FLI) and, on the other hand, a vector expressing an antisense directed against the AUG of zyxin was introduced into a non-tumorigenic line (NIH3T3). The results obtained show that the restoration of the expression of zyxin in tumoral lines (FIG. 6) makes it possible to considerably diminish the tumorigenic power of these cells. Given that zyxin is a very highly conserved protein (97% of homology between humans and mice), the sequence difference between human and murine zyxin is very probably not at the origin of this phenomenon.

Moreover, selective inhibition of the expression of zyxin in non-tumorigenic NIH3T3 fibroblasts (FIG. 9) leads to the malignant transformation of these cells. Additional data indicate that there exists a direct link between the effect on malignant transformation and the level of expression of zyxin. Certain mice having received injections of EWS-FLI cells overexpressing human zyxin developed tumors. Analysis of these tumors showed that the vector enabling expression of zyxin was always present; in contrast, the RNA coding for human zyxin was not detected (experiment not presented). Comparative analysis of the microarrays between the NIH3T3 parent line and the clones derived from it which underexpress zyxin was not able to detect modifications of expression of other microfilament proteins such as α-actinin, tropomyosine, actin or vinculin, or the proteins of the cytoskeleton such as tubulin or vimentin (FIG. 10).

The results obtained show clearly that there exists a direct link between the level of expression of zyxin and the malignant transformation although the mechanism was not established. The actin cytoskeleton in association with the plasma membrane is organized in specialized domains capable of assuring specific functions in motility (lamellipodium), adherence (adhesion plaque) or interactions between cells (junction plate). Fluorescence microscopy (FIG. 3) shows that the diminution of expression of zyxin in the cells is manifested by the putting in place of a domain of the lamellipodium to the detriment of the adhesion plaques and junctions. The actin filaments play an essential role in the formation and maintenance of these different domains, particularly dynamic, which require the concomitant formation and disassembly of different structures. Under these conditions, it is evident that the deregulation of one of the elements intervening in these structures is sufficient to disturb the entire system. Zyxin is an essential structural component of the microfilaments and adhesion plaques and it influences the organization of these microfilaments (Crawford, A. W. et al.; 1992) as well as the properties of cell adhesion (Macalma, T et al.; 1996) and cellular motility (Drees, B. E., et al.; 1999). It is thus possible that the modifications of the structure of the cells observed when zyxin is underexpressed are sufficient to make these cells tumorigenic. The modifications of the adherence parameters of the cells are manifested by a modification of the cell/environment interactions with as consequence a reprogramming of the expression of key genes responsible for phenotypic modifications. It was thus observed that the acquisition of the tumor phenotype induced in NIH3T3 cells by a diminution in the zyxin protein leads to a modification of the expression of genes characterizing the invasive phenotype, notably an underexpression of the genes TIMP2 and TIMP3 and protease nexin-1 (PN-1) and an overexpression of the urokinase plasminogen activator (FIG. 10). Consequently, these genes and their expression product can be used, as described above with regard to zyxin, for the preparation of pharmaceutical compositions useful for the diagnosis, prevention or treatment of cancers, or for the screening of compounds.

1-47. (canceled)

48. A method of treating a cancer having a zyxin gene expression/abnormality selected from the group consisting of hepatocarcinomas, mesenchymal tumors, neuroectodermal cancer, Ewing's sarcoma, melanoma, and malignant hemopathies associated with chromosomal anomalies of region of 7q34/q35 of zyxin gene, comprising administering a therapeutically effective amount of a composition comprising an active agent which stabilizes an actin network of a cellular cytoskeleton and wherein said active agent is dolastatin 11, to a patient in need thereof.

49. The method according to claim 48, wherein said cancer is Ewing's sarcoma.

50. A method of treating a cancer having a zyxin gene expression/abnormality selected from the group consisting of hepatocarcinomas, mesenchymal tumors, neuroectodermal cancer, Ewing's sarcoma, melanoma, and malignant hemopathies associated with chromosomal anomalies of region of 7q34/q35 of zyxin gene, comprising:
a) identifying a subject having said cancer underexpressing a zyxin gene;
b) administering a therapeutically effective amount of a pharmaceutical composition to the subject, the composition comprising:
(i) dolastatin 11 and
(ii) a pharmaceutically acceptable carrier.

51. The method according to claim 50, wherein said cancer is selected from the group consisting of Ewing's sarcoma and melanoma.

52. The method according to claim 51, wherein said cancer is Ewing's sarcoma.


SEQUENCE LISTING (4 sequences)

SEQ ID NO: 1 - DNA, 19 bp, Artificial Sequence
  Description of Artificial Sequence Primer
agatatcctgtttggccat

SEQ ID NO: 2 - DNA, 20 bp, Artificial Sequence
  Description of Artificial Sequence Primer
gccgtgcatcatcctgactg

SEQ ID NO: 3 - DNA, 20 bp, Artificial Sequence
  Description of Artificial Sequence Primer
ctgttcctgaccttgatctg

SEQ ID NO: 4 - DNA, 24 bp, Artificial Sequence
  Description of Artificial Sequence Primer
cggcccggccatggcggccccccg